(19) United States
(12) Patent Application Publication
(10) Pub. No.: US 2002/0087213 A1
(43) Pub. Date: Jul. 4, 2002

(54) Total joint replacements using magnetism to control instability

(76) Inventor: Morton Bertram III, Naples, FL (US)

Correspondence Address: John G. Posa, Gifford, Krass, Groh, Suite 400, 280 N. Old Woodward Ave., Birmingham, MI, 48009, US

(21) Appl. No.: 10/087,052

(22) Filed: Oct. 18, 2001

Related U.S. Application Data
(60) Provisional application No. 60/241,401, filed on Oct. 18, 2000.

(51) Int. Cl. A61F 2/30

(52) U.S. Cl. 623/18.12

(57) ABSTRACT

Magnetic force fields are used to control the instability of joint-replacement situations. Prosthetic components according to the invention are fabricated with opposite-polarity magnets on either side of the joint surface, so that an inherent stability is conferred to the joint. The magnets are of sufficient strength so that dislocation or uncoupling of the components would be very difficult, but not impossible. The forces do, however, allow motion between the bearing surfaces, without increasing friction between the joint surfaces. The approach is applicable to various artificial joint situations, including the hip, shoulder, ankle, elbow, knee, and smaller joints. As opposed to pairs of magnets which attract one another, in certain applications the magnets may be arranged so as to repel one another such that the components actually touch little or not at all, but function as they do now. Such an approach would represent the ultimate application of this technology, namely, the frictionless bearing surface for joint replacement prostheses.

### REFERENCE TO RELATED APPLICATION

This application claims priority from U.S. Provisional Patent Application Serial No. 60/241,401, filed Oct. 18, 2000, the entire contents of which are incorporated herein by reference.

### BACKGROUND OF THE INVENTION

Total joint replacement has become a common procedure in the United States and elsewhere in the world. Arthroplasty of the hip, knee, shoulder, ankle, and elbow are the most frequent applications. Smaller joints are replaced as well.

Unfortunately, instability continues to be a problem. The most common reasons for instability of joint replacements is muscular weakness, which makes the joint less stable and more prone to dislocation or uncoupling. Other reasons include decreases in mental acuity, malposition of components, and alcohol.

It is estimated that $75 million is now spent in the United States annually in conjunction with total hip replacement instability. This includes costs associated with repeat surgeries and hospitalizations to correct instability problems. Surgical methods for controlling instability are not entirely effective. The current solution is bracing, repeat surgery to correct any malposition of components, and muscle advancement or retensioning.

A certain percentage of patients have unsolvable problems, necessitating drastic measures to address their situations. This usually involves performing a Girdlestone procedure, which involves removal of the prosthesis altogether, leaving nothing in the joint. Frequently this results in a “flail” limb, with significant functional deficits. An inability to solve these problems, not infrequently, leads to litigation because of the frustration felt by the patient. These, in turn, lead to additional costs, exascerbating the problem.

Although certain inventions have been disclosed and patented wherein magnetism is used in joint-replacement surgery, none so far have been specifically directed to solving the problems associated with instability. U.S. Pat. No. 5,879,386, entitled “Magnetic Prosthetic System” uses magnetism to hold the bones apart during articulation to reduce friction. U.S. Pat. No. 5,571,195 to Johnson, entitled “Prosthesis For An Artificial Joint Having A Wear Particle Collection Capability” utilizes magnetism to collect metal wear particles. U.S. Pat. No. 5,092,320 to Maurer uses magnets (70) to secure a knee brace to the leg of a wearer. U.S. Pat. No. 4,216,548 to Kraus utilizes magnets and electromagnetism to stimulate bone growth/ingrowth. U.S. Pat. No. 3,140,712, entitled “Articulated Joint,” for example, artificially duplicates the vacuum or suction [of a joint] by means of a magnetizable metal cup.

U.S. Pat. Nos. 4,743,264 and 4,781,720 to Sherva-Parker use magnetic traction to retain external prosthetic devices, i.e., amputation apparatus. U.S. Pat. No. 5,062,855 to Rincoe teaches the use of magnetism to control an artificial limb. U.S. Pat. No. 5,507,835 to Jore discloses a first embodiment wherein magnetic fixtures are used to hold an external prosthesis in place, and a second embodiment wherein repelling magnetic forces are used to hold bones apart.

European patents EP0578969A3 and EP0578969B1 disclose magnet arrangements for a prosthesis in particular, for a dental prosthesis. In addition, European patents EP0533384A1 and EP0533384B1 disclose a prosthesis for use with an ossicular chain to allow a magnetic to be coupled to the ossicular chain. European patents EP0638293A1 and EP0638293B1 show a device for positioning a magnet in a prosthesis, apparently a dental prosthesis as well.

### SUMMARY OF THE IVNENTION

The instant invention solves problems evident in the current art by employing magnetic force fields to control the instability of joint-replacement operations. According to the preferred embodiment, prosthetic components according to the invention are fabricated with opposite-polarity magnets on either side of the joint surface, so that an inherent stability is conferred to the joint. The magnets are of sufficient strength so that dislocation or uncoupling of the components would be very difficult, but not impossible. The forces would, however, allow motion between the bearing surfaces, without increasing friction between the joint surfaces. The approach is applicable to various artificial joint situations, including the hip, shoulder, ankle, elbow, knee, and smaller joints.

### BRIEF DESCRIPTION OF THE DRAWINGS

FIG. 1 is an exploded view of a total hip system, showing how the components are assembled;

FIG. 2 is a drawing which shows how the head of a femoral component fits on the trunion of the prosthesis;

FIG. 3 is a drawing which shows an acetabular component with an apical hole;

FIG. 4 is a drawing which shows the acetabular component from the concave side;

FIG. 5 is a drawing which shows a two-piece modular arrangement wherein a ceramic liner fits into the acetabular shell;

FIG. 6 is a cross-sectional view of the acetabular shell and the liner installed therein;

FIG. 7 is a drawing which illustrates a metal-on-metal hip replacement system;

FIG. 8 illustrates the so-called “pistoning effect,” wherein, during the swing phase of gait, certain joint components briefly separate; and

FIG. 9 illustrates a shoulder replacement application of the invention.

### DETAILED DESCRIPTION OF THE INVENTION

The invention will now be described in detail with reference to the accompanying figures. As discussed above, although the embodiments will be described in conjunction with a total hip replacement, it will be apparent to those of skill that the approach is applicable to alternate joint situations, including the shoulder, elbow, knee, and smaller joints.

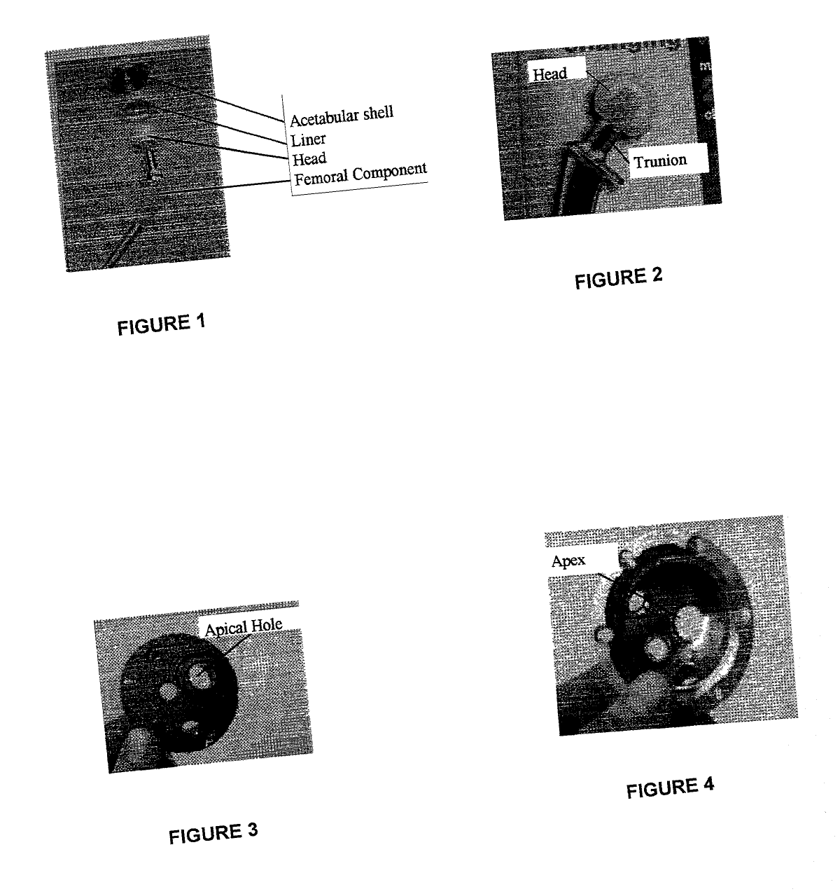

FIG. 1 is an exploded view of a total hip system, showing how the components are assembled. With respect to hip replacement, in the preferred embodiment a first magnet is positioned on the pole of the acetabular component, and a second magnet, oriented to be attractive to the first magnet, is supported on the trunion of the femoral component. For the sake of convenience, the second magnet may be disposed in the apical hole typically used for the insertion of instrumentaton.

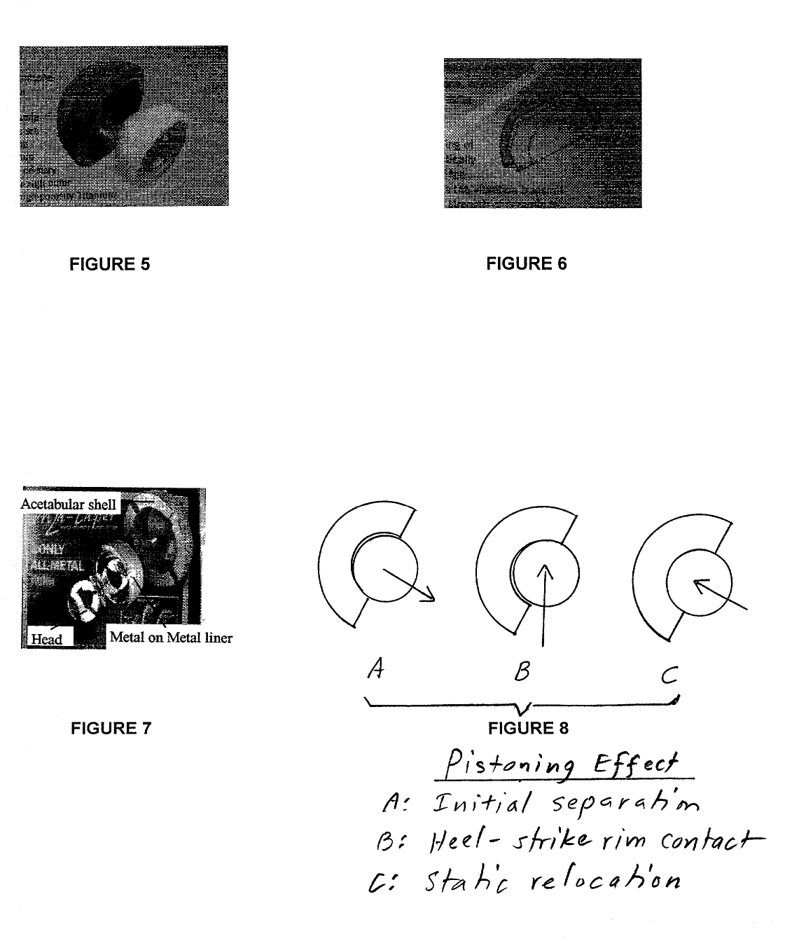

FIG. 2 is a drawing which shows how the head of a femoral component fits on the trunion of the prosthesis. FIG. 3 shows the pole of the acetabular component. FIG. 4 shows the acetabular component from the concave side. FIG. 5 shows a two-piece modular arrangement wherein a liner, in this case ceramic, fits into the acepabular shell. FIG. 6 is a cross-sectional view of the acetabular shell and the liner installed therein.

The magnets according to the invention are incorporated into the acetabular component during the manufacturing process to prevent oxidation or other deterioration of the surfaces. Since magnets only work effectively within a certain range or “air gap,” beyond which the magnets exhibit no attraction, the magnets may form part of the total joint implants without fear of attraction from very strong magnetic fields of the type used with medical and industrial instrumentation. The invention is also fully compatible with metal-on-metal hip replacements of the type depicted in FIG. 7. This would eliminate problems with the air-gap phenomenon.

Another important aspect of the invention addresses the “pistoning effect,” wherein, during the swing phase of gait, certain joint components briefly separate. When the components recouple or “relocate,” the effect tends to increase the wear of the surfaces. The effect is illustrated in FIG. 8. It is believed that his phenomenon accounts for the fact that higher wear rates are seen in vivo, as compared to in vitro studies. The magnetic attraction provided by this invention between the two surfaces would minimize if not eliminate this problem. Wear rates would accordingly be lowered, improving the longevity of the implants.

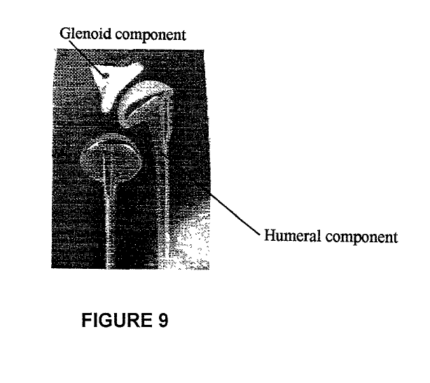

FIG. 9 illustrates a shoulder replacement application of the invention. A magnet is embedded either in the polyethylene (plastic) component(s) or in the metal backing of the of the glenoid component. As opposed to pairs of magnets which attract one another, in certain applications the magnets may be arranged so as to repel one another such that the components actually touch little or not at all, but function as they do now. Such an approach would represent the ultimate application of this technology, namely, the frictionless bearing surface for joint replacement prostheses.

I claim:

1. Reduced dislocation total joint replacement apparatus, comprising:
a first prosthetic component having a first bearing surface;
a second component element having a second bearing configured to co-act with the first bearing surface; and
a magnet associated with one or both of the first and second bearing surfaces,
wherein the magnitude of the magnetic attraction minimizes dislocation or uncoupling of the components while allowing relative movement of the bearing surfaces.

2. The apparatus of claim 1, including:
a first magnet associated with the first bearing surface; and
a second magnet associated with the second bearing surface, wherein the second magnet is oriented so as to attract the first magnet.

3. The apparatus of claim 1, wherein the components are associated with a total hip, knee, shoulder, ankle or elbow replacement.

4. The apparatus of claim 3, wherein the components are associated with a total hip replacement, and one of the magnets is disposed in the apical hole of the acetabular component generally used for the insertion of instrumentation.

5. Reduced dislocation total hip replacement apparatus, comprising:
an acetabular component having a first bearing surface;
a proximal femoral component having a second bearing configured to co-act with the first bearing surface; and
a magnet associated with one or both of the first and second bearing surfaces, wherein the magnitude of the magnetic attraction minimizes dislocation or uncoupling of the components while allowing relative movement of the bearing surfaces.

6. The apparatus of claim 5, including:
a first magnet associated with the first bearing surface; and
a second magnet associated with the second bearing surface, wherein the second magnet is oriented so as to attract the first magnet.

7. The apparatus of claim 5, wherein:
the acetabular component includes an apical hole generally used for the insertion of instrumentation; and
one of the magnets is disposed in the apical hole.
